(19) United States
(12) Patent Application Publication
(10) Pub. No.: US 2017/0112741 A1
(43) Pub. Date: Apr. 27, 2017

(54) AGENT FOR AMELIORATING SKIN SYMPTOM, HAIR GROWTH AGENT OR SLIMMING AGENT

(71) Applicant: GOLDCREST MEDICINE INSTITUTE CO., LTD., Tokyo (JP)

(72) Inventor: Kazuo TORII, Machida-shi (JP)

(73) Assignee: GOLDCREST MEDICINE INSTITUTE CO., LTD., Tokyo (JP)

(21) Appl. No.: 15/302,925

(22) PCT Filed: Apr. 8, 2015
(86) PCT No.: PCT/JP2015/060916

§ 371 Date: Dec. 7, 2016

(30) Foreign Application Priority Data
Apr. 9, 2014 (JP) 2014-079959

(51) Int. Cl. A61K 8/49; A61Q 19/02; A61K 8/02

(52) U.S. Cl. CPC A61K 8/4926; A61K 8/0216; A61Q 19/02; A61K 2800/92

(57) ABSTRACT

To provide a novel agent for ameliorating a skin symptom such as pigmented macules or ephelides, a novel hair growth agent, and a novel slimming agent. The agent for ameliorating a skin symptom, the hair growth agent, or the slimming agent contains a dopamine agonist as an active ingredient, in which the skin symptom is selected from pigmented macules, periorbital dark circles, darkish skin, black darkish skin, moles, ephelides, birth marks, and seborrheic keratosis.

### FIELD OF THE INVENTION

The present invention relates to an agent for ameliorating a skin symptom such as pigmented macules or ephelides, a hair growth agent, and a slimming agent.

### BACKGROUND OF THE INVENTION

Unlike fatal disorders such as cancer, skin symptoms including periorbital dark circles, darkish skin, black darkish skin, pigmented macules, ephelides, moles, birth marks, and seborrheic keratosis, which occur on the face or the body, are hardly life-threatening. However, from the cosmetic point of view, it is strongly desired to ameliorate those skin symptoms.

Furthermore, a skin symptom such as senile plaque, chloasma, or seborrheic keratosis is a symptom that is unfavorable in terms of appearance, and it is a disease which needs to be treated.

Regarding those skin symptoms, it is known that, as melanin, which is a coloration material, is excessively produced in the melanocytes in the living body and the coloration material is accumulated, the skin of the cheek, back of hand, and arm gradually turns into a dark color and the above skin symptoms is caused (Non Patent Literature 1).

Aging progresses accompanied by a decrease in melanocytes, the decrease becomes apparent after 40 years of age, and the rate of decrease is 10 to 20% per decade. In particular, it is known that, regarding the scalp, hair color gradually becomes gray and senile plaque gradually increases on the skin (Non Patent Literature 1).

Moles are small-size pigmented spots occurring on the skin, and the Japanese have moles mostly with black color or dark brown color. Most of the moles are benign. When pigment cells called melanocytes in the living body grow to form a cluster, the resulting mass corresponds to a mole. Moles are commonly observed. When the moles look unusual, grow to a large size, show a change in color or appearance, or look differently from other moles, it is better to consult with a doctor (Non Patent Literature 2).

Furthermore, in seborrheic keratosis, dark brown or black lesions of the skin are elevated, and in many cases the lesions are present, for example, on the chest area, back, back of hand, or arm (Non Patent Literature 3).

In recent years, as a therapy for pigmented macules such as senile plaque, a laser therapy using alexandrite laser (755 nm) or ruby laser (694 nm) or a cryotherapy using liquid nitrogen is applied. However, as a side effect of the laser therapy, there are problems of, for example, occurrence of pain, postinflammatory hyperpigmentation, redness, scar, or hypopigmentation. Furthermore, as a side effect of the cryotherapy, there is a pain or hypopigmentation with prolonged exposure (Non Patent Literature 4).

In the specification of U.S. Pat. No. 5,279,834, components of cosmetics or medicinal agents are suggested and a product for application in which hydroquinone and kojic acid are used as skin whitening components is described (Patent Literature 1). As one of the materials that are most effective for the removal of skin coloration pigment, hydroquinone is known. It is reported that a strong side effect is caused by the use of hydroquinone (Non Patent Literature 5). Furthermore, arbutin as a hydroquinone type chemical substance and kojic acid have been used for the treatment of pigmented macules (Patent Literature 2 and Patent Literature 3). However, their effects are not sufficient. Furthermore, tranexamic acid has been used for the treatment of chloasma, either by oral administration or by external application (Patent Literature 4). However, their effects are not sufficient.

As a treatment method for moles, birth marks, and seborrheic keratosis, for example, a cryotherapy, a carbonate gas laser method, or surgery excision is carried out. However, as there is a problem of remaining pain or scars (Non Patent Literature 4), it is desired to develop a treatment which uses an orally administered agent with higher safety.

Along with aging, a lot of men experience a loss of hair from the center of the head, and their hairs become thinner as they grow old. As such, a means for preventing or ameliorating the hair loss or thinning hair by using, for example, male hormone is desired.

As a hair growth agent, finasteride, which is a type 2 5α reductase antagonist, is widely used. Minoxidil, carpronium chloride or the like is also used. However, finasteride has a side effect such as decreased libido (Non Patent Literature 6). Furthermore, minoxidil has a side effect such as hypotensive action, and carpronium chloride has a side effect such as rash on the scalp.

In addition, as obesity causes various lifestyle related diseases, high attention is paid to slimming in recent years, and many food products and the like, for example, for promoting lipid combustion are being developed.

Increase in the intracellular cAMP is suitable for the purpose of slimming (that is, health management for weight loss by diet therapy or exercises). In fact, the intracellular cAMP can release glycerol via adipocyte lipase (HSL). As the intracellular lipids are consumed accordingly, the cell volume can be reduced. It is also known that the slimming may progress according to the mechanism in which intracellular Ca²⁺ is used (Patent Literature 5).

### CITATION LIST

### Patent Literature

Patent Literature 1: U.S. Pat. No. 5,279,834
Patent Literature 2: JP-A-S60-16906
Patent Literature 3: JP-A-S53-3538
Patent Literature 4: JP-A-2010-229117
Patent Literature 5: US 2006/011413 A1

### Non Patent Literature

Non Patent Literature 1: John Woodruff, Feature: Correcting skin tone, Published SPC Magazine; Autumn 2008.
Non Patent Literature 2: Moles, Medline Plus, U.S. National Library of Medicine, 26 Mar. 2014, National Institutes of Health. Date last updated: 26 Mar. 2014. Topic last Reviewed: 5 Mar. 2014. URL of this page;
http://www.nlm.gov/medlineplus/moles.html
Non Patent Literature 3: Seborrheic keratosis, Medline Plus, U.S. National Library of Medicine, 20 Nov. 2012, National Institutes of Health. Update Date; Nov. 20, 2012. URL of this page: http//www.nlm.nih.gov/medlineplus/ency/artile/000884.html
Non Patent Literature 4: S. Plensdore and J. Martiner, Am. Fam. Physician, 2009 Jan. 15, 79 (2), 109-116.
Non Patent Literature 5: H. Ando, Mary S. Matsui and M. Ichihashi, Int. J. Mol. Sci, 2100, 11, 2566-2575; Quasi-drugs developed in Japan for the prevention or treatment of hyperpigmentary disorders.
Non Patent Literature 6: Drugs.com, Finastride, Issue Date; Feb. 4, 2015, Database Edition 15.1.1.002, Copyright (c) 2015 Wolters Kluwer Health, Inc.

### SUMMARY OF THE INVENTION

### Problems to be Solved by the Invention

An object of the present invention is to provide a novel agent for ameliorating a skin symptom such as pigmented macules, moles, or seborrheic keratosis, a novel hair growth agent, and a novel slimming agent.

### Means for Solving the Problems

Under such circumstances, to develop an agent for ameliorating a skin symptom such as senile plaque, the present inventor investigated various combinations of many receptors that are present in melanocytes and a dopamine agonist and the like. As a result, the present inventor found that, by administering a dopamine agonist which is widely used as a therapeutic agent for Parkinson's disease, a skin symptom including pigmented macules such as senile plaque and chloasma, moles, seborrheic keratosis, periorbital dark circles, darkish skin, black darkish skin, ephelides, and birth marks is significantly improved, the black color of hairs becomes darker, thinning hairs are improved, that is, a hair growth effect is obtained, and also an effect as a slimming agent is exhibited. Accordingly, the present invention has been completed.

Namely, the present invention provides the following [1] to [20].

[1] An agent for ameliorating a skin symptom, a hair growth agent, or a slimming agent, comprising a dopamine agonist as an active ingredient, wherein the skin symptom is selected from the group consisting of pigmented macules, periorbital dark circles, darkish skin, black darkish skin, moles, ephelides, birth marks, and seborrheic keratosis.

[2] The agent for ameliorating a skin symptom, the hair growth agent, or the slimming agent according to [1], wherein the dopamine agonist is one or more kinds selected from the group consisting of cabergoline, pergolide, bromocriptine, talipexole, pramipexole, ropinirole, rotigotine, apomorphine, terguride, aripiprazole, and a salt thereof.

[3] The agent for ameliorating a skin symptom, the hair growth agent, or the slimming agent according to [1] or [2], which is an orally administered agent.

[4] The agent for ameliorating a skin symptom, the hair growth agent, or the slimming agent according to any one of [1] to [3], wherein the dopamine agonist is cabergoline and a dose thereof is 0.1 to 3 mg/week.

[5] The agent for ameliorating a skin symptom, the hair growth agent, or the slimming agent according to any one of [1] to [3], wherein the dopamine agonist is cabergoline and a dose thereof is 0.1 to 0.5 mg/week.

[6] Use of a dopamine agonist for producing an agent for ameliorating a skin symptom, a hair growth agent, or a slimming agent, wherein the skin symptom is selected from the group consisting of pigmented macules, periorbital dark circles, darkish skin, black darkish skin, moles, ephelides, birthmarks, and seborrheic keratosis.

[7] The use according to [6], wherein the dopamine agonist is one or more kinds selected from the group consisting of cabergoline, pergolide, bromocriptine, talipexole, pramipexole, ropinirole, rotigotine, apomorphine, terguride, aripiprazole, and a salt thereof.

[8] The use according to [6] or [7], wherein the agent for ameliorating a skin symptom, the hair growth agent, or the slimming agent is an orally administered agent.

[9] The use according to any one of [6] to [8], wherein the dopamine agonist is cabergoline and a dose thereof is 0.1 to 3 mg/week.

[10] The use according to any one of [6] to [8], wherein the dopamine agonist is cabergoline and a dose thereof is 0.1 to 0.5 mg/week.

[11] A dopamine agonist for use in skin symptom amelioration, hair growth, or slimming, wherein the skin symptom is selected from the group consisting of pigmented macules, periorbital dark circles, darkish skin, black darkish skin, moles, ephelides, birth marks, and seborrheic keratosis.

[12] The dopamine agonist according to [11], wherein the dopamine agonist is one or more kinds selected from the group consisting of cabergoline, pergolide, bromocriptine, talipexole, pramipexole, ropinirole, rotigotine, apomorphine, terguride, aripiprazole, and a salt thereof.

[13] The dopamine agonist according to [11] or [12], which is used by oral administration.

[14] The dopamine agonist according to any one of [11] to [13], wherein the dopamine agonist is cabergoline and a dose thereof is 0.1 to 3 mg/week.

[15] The dopamine agonist according to any one of [11] to [13], wherein the dopamine agonist is cabergoline and a dose thereof is 0.1 to 0.5 mg/week.

[16] A method for ameliorating a skin symptom, a hair growth method, or a slimming method, comprising administering an effective amount of a dopamine agonist, wherein the skin symptom is selected from the group consisting of pigmented macules, periorbital dark circles, darkish skin, black darkish skin, moles, ephelides, birth marks, and seborrheic keratosis.

[17] The method according to [16], wherein the dopamine agonist is one or more kinds selected from the group consisting of cabergoline, pergolide, bromocriptine, talipexole, pramipexole, ropinirole, rotigotine, apomorphine, terguride, aripiprazole, and a salt thereof.

[18] The method according to [16] or [17], wherein the dopamine agonist is orally administered.

[19] The method according to any one of [16] to [18], wherein the dopamine agonist is cabergoline and an effective amount thereof is 0.1 to 3 mg/week.

[20] The method according to any one of [16] to [18], wherein the dopamine agonist is cabergoline and an effective amount thereof is 0.1 to 0.5 mg/week.

### Effects of the Invention

The agent for ameliorating a skin symptom of the present invention can alleviate a skin symptom, for example, pigmented macules such as senile plaque or chloasma, periorbital dark circles, darkish skin, black darkish skin, moles, ephelides, birth marks, and seborrheic keratosis, and, for example, can treat an elevated lesion, reduce or resolve the symptom, and ameliorate the skin color change so that the skin color can be lightened and brought back to the original color.

Furthermore, the hair growth agent of the present invention can darken the hair color, prevent hair loss, and improve thinning hair. Furthermore, the slimming agent of the present invention can reduce the body weight based on intracellular lipid degradation and it is useful for the health management.

### BRIEF DESCRIPTION OF THE DRAWINGS

FIG. 1 is a photographic image illustrating the frontal region of the head of patient A on Sep. 16, 2010.

FIG. 2 is a photographic image illustrating the occipital region of the head of patient A on Sep. 16, 2010.

FIG. 3 is a photographic image illustrating the frontal region of the head of patient A on Feb. 27, 2014.

FIG. 4 is a photographic image illustrating the occipital region of the head of patient A on Feb. 27, 2014.

### MODES FOR CARRYING OUT THE INVENTION

The active ingredient of the agent for ameliorating a skin symptom, the hair growth agent, and the slimming agent of the present invention is a dopamine agonist. The dopamine receptors are classified into D1-like receptors and D2-like receptors. However, for the agent for ameliorating a skin symptom and the hair growth agent of the present invention, a D2-like receptor stimulating agent is preferable. On the other hand, for the slimming agent of the present invention, a D1-like receptor stimulating agent is preferable. The dopamine agonist such as cabergoline acts on both of D1-like receptors and D2-like receptors.

As the dopamine agonist, one or more kinds selected from the group consisting of cabergoline, pergolide, bromocriptine, talipexole, pramipexole, ropinirole, rotigotine, apomorphine, terguride, aripiprazole, and a salt thereof are preferable. Herein, examples of the salt include a salt of a mineral acid such as hydrochloric acid, sulfuric acid, or nitric acid, and a salt of an organic acid such as acetic acid, oxalic acid, citric acid, mesylic acid, or tosylic acid.

More specific examples thereof include one or more kinds selected from the group consisting of cabergoline, pergolide mesylate, bromocriptine mesylate, talipexole hydrochloride, pramipexole hydrochloride, ropinirole hydrochloride, rotigotine, apomorphine hydrochloride, terguride, and aripiprazole.

Those dopamine agonists are known as a therapeutic agent for Parkinson's disease, prolactin-producing pituitary adenoma, or hyperprolactinemic anovulation. However, nothing is known regarding the effect of ameliorating the skin symptoms, the hair growth effect, or the slimming effect described above. Although the reason for a dopamine agonist to have an ameliorating effect for skin symptoms such as pigmented macules and moles remains unclear, it is considered that the effect is based on the prevention of melanin production in melanocytes. Namely, it is considered that the dopamine receptor plays a certain role in the process in melanocytes where DOPA is synthesized from tyrosine by tyrosinase in melanosomes and black eumelanin is produced via dopaquinone.

By the administration of a dopamine agonist, the effect of ameliorating skin symptoms selected from the group consisting of pigmented macules, periorbital dark circles, darkish skin, black darkish skin, moles, ephelides, birthmarks, and seborrheic keratosis, and the hair growth effect can be obtained. Herein, the effect of ameliorating skin symptoms includes an effect of preventing the progress of the skin symptoms, a whitening effect, and an effect of ameliorating skin tone.

Factors for determining human skin tone include melanin, carotene, and hemoglobin, and melanin has a particularly high influence. As one of the skin pigmentation disorders, there may be mentioned hyperpigmentation, and examples thereof include pigmented macules, ephelides, darkish skin, and black darkish skin.

The pigmented macules include common pigmented macules, senile plaque, and chloasma. Furthermore, the darkish skin may be caused by ultraviolet rays, skin dryness, or skin aging due to aging. The black darkish skin may be caused by friction with underwear or clothes. The periorbital dark circles mean a darkish part under eyes, and examples thereof include blue circles caused by lack of sleeping and brown circles caused by ultraviolet rays or friction.

Moles are melanocytic nevi. Examples of a nevus cell nevus include junctional nevus, compound nevus, intradermal nevus, Spitz nevus, Clark nevus, pigmented spot, and melanotic macule, and examples of a dermal melanocytic nevus include blue nevus, nevus of Ota, acquired bilateral nevus of Ota-like macule, and Mongolian spot. Furthermore, the seborrheic keratosis is a skin disease which is also referred to as verruca senilis.

The hair growth effect includes both the hair loss prevention and hair growth promotion. Furthermore, the effect of the hair growth agent of the present invention is not different between males and females, and it can be applied to any type of alopecia. Furthermore, the hair growth agent of the present invention is particularly useful for head hair growth.

Although the mechanism of the slimming effect remains unclear, it is considered that the mechanism is related with the increase of cAMP in cells, in particular, in adipocytes.

The agent for ameliorating a skin symptom, the hair growth agent, and the slimming agent of the present invention can be blended with other components. Examples of the components which may be used in combination include an antihistamine drug, an antiallergic drug, an antimicrobial drug, an antifungal drug, an antivirus drug, a steroid oral medicine, an immunosuppressive drug, a biological preparation, an anti-malignant tumor drug, a vitamin drug, vitamin A such as retinoid, and a Chinese herbal medicine.

Examples of the antihistamine drug include, as a first generation antihistamine drug, diphenhydramine hydrochloride, d-chlorphenylamine maleate, hydroxyzine, homochlorcyclizine hydrochloride, clemastine fumarate, and ciproheptadine hydrochloride hydrate. Examples of the antihistamine drug further include, as a second generation antihistamine drug, ketotifen fumarate, azelastine hydrochloride, oxatomide, and emedastine fumarate. Furthermore, as a third generation antihistamine drug, for example, fexofenadine hydrochloride, olopatadinehydrochloride, epinastinehydrochloride, ebastine, cetirizine hydrochloride, loratadine, or levocetirizine hydrochloride can be effectively used.

As an antiallegric drug, tranilast, sodium cromoglicate, and suplatast tosilate can be used.

Examples of the antimicrobial drug include (1) an antimicrobial drug which exhibits an antimicrobial effect by inhibiting the synthesis of cell wall, such as a penicillin-based, cephem-based, monobactam-based, carbapenem-based, penem-based, fosfomycin-based, or polypeptide-based antimicrobial drug, (2) an aminoglycoside-based antimicrobial drug which exhibits an antimicrobial effect by inhibiting the protein synthesis, (3) a new quinolone-based antimicrobial drug which exhibits an antimicrobial effect by inhibiting the synthesis of nucleic acid, and (4) an ST mixture which exhibits an antimicrobial effect by inhibiting the synthesis of folic acid, and an injection solution for deep mycosis (for example, itraconazole, fluconazole, micafungin, or voriconazole). Furthermore, an antifungal drug such as terbinafine hydrochloride, an antimicrobial drug which exhibits a bacteriostatic effect by inhibiting the protein synthesis, such as a tetracycline-based, chloramphenicol-based, macrolide-based, or lincomycin-based antimicrobial drug, and the like, may also be used.

Furthermore, an orally administered antifungal drug such as itraconazole, terbinafine hydrochloride, griseofulvin, amphotericin, nystatin, flucytosine, or miconazole can also be used.

An anti-virus drug such as aciclovir, valacyclovir, vidarabine, famciclovir, or ganciclovir may also be used.

As a steroid preparation, hydrocortisone, prednisolone, methylprednisolone, dexamethasone, betamethasone, and the like, may be used. For example, when it is difficult to reduce the amount of steroids, an immunosuppressive agent such as cyclosporine, azathioprine, methotrexate, or cyclophosphamide may be used. A biological preparation using monoclonal antibodies such as infliximab, adalimumab, rituximab, etanercept, alefacept, and ustekinumab, which has fewer side effects than anti-malignant tumor drug or immunosuppressive drug, may also be used. Examples of the anti-malignant tumor drug which may be used include an alkylating agent such as cyclophosphamide, dacarbazine, nimustine, temozolomide, or cisplatin, an antimetabolite such as methothexate or fluorouracil, a microtubule inhibitor such as vincristine, docetaxel, or paclitaxel, an antitumuor antimicrobial agent such as doxorubicin, bleomycin, mitomycin C, epirubicin, or pirarubicin, and a topoisomerase inhibitor such as etoposide.

Furthermore, as the vitamin drug, vitamin B₂, niacin, biotin, and vitamin C may be used. Retinoid may also be used.

Examples of the Chinese herbal medicine include Orengedokuto, Byakkokaninjinto, Jizusoippo, Unseiin, Tokiinshi, Seijobofuto, Jumihaidokuto, Keigairengyoto, Keishibukuryogan, Tokakujokito, Tokishakuyakusan, Inchingoreisan, Shofusan, Saikozai, Inchinkoto, Saikokaryukotsuboreito, Hangekobokuto, Keishikaryukotsuboreito, Kamishoyosan, Hangeshashinto, Unkeito, Shimotsuto, and Eppikajutsuto.

The agent for ameliorating a skin symptom, the hair growth agent, and the slimming agent of the present invention can be used as a pharmaceutical product, a quasi-pharmaceutical product, a cosmetic product, or a functional food product.

The agent for ameliorating a skin symptom, the hair growth agent, and the slimming agent of the present invention can be administered by, for example, oral administration, intravenous administration, transdermal administration, or rectal administration. However, the oral administration and transdermal administration (including administration via the scalp) are preferable.

Examples of the formulation for oral administration include a tablet, a capsule, a granule, a powder, a syrup, a suspension, and a liquid. To prepare those formulations for oral administration, for example, a vehicle, a lubricant, a disintegrant, a binding agent, a corrigent, or a flavor may be blended.

Examples of the formulation for transdermal administration include, in addition to an ointment, a gel, a cream, an emulsion, and a liquid, a plaster preparation such as a plaster, a cataplasm, or a tape preparation. To prepare those formulations, for example, an adhesive base may be blended in addition to an ointment base, a gel base, an oil agent, a surfactant, and a gelling agent.

Examples of the formulation for injection include a solution, a suspension, an emulsion, and a solid formulation which is used after being dissolved or suspended in a solvent. Examples of the solvent which may be used include distilled water for injection, physiological saline, plant oil, propylene glycol, polyethylene glycol, and alcohols such as ethanol, and a combination thereof. Furthermore, the injection may include, for example, a stabilizing agent, a dissolution aid (for example, glutamic acid, asparaginic acid, or polysorbate 80 (registered trademark)), a suspending agent, an emulsifying agent, a soothing agent, a buffering agent, or a preservative. They are sterilized in the final process or prepared or adjusted by a sterile operation method. Alternatively, the injection may be prepared as a sterile solid preparation, for example, a freeze-dried product may be prepared, sterilized before use or dissolved in sterile distilled water or other solvent for injection, and then used.

The agent for ameliorating a skin symptom, the hair growth agent, and the slimming agent of the present invention may contain the dopamine agonist in an amount of 0.001 to 50% by mass, and preferably 0.01 to 20% by mass.

The use amount of each of the agent for ameliorating a skin symptom, the hair growth agent, and the slimming agent of the present invention is, in terms of a dopamine agonist, preferably 0.05 mg to 100 mg, and more preferably 0.05 mg to 20 mg per day for an adult.

The number of doses is generally affected by the half life of a pharmaceutical, and in the case of a pharmaceutical with a short half life of about 6 hours, it is preferably 1 to 3 times per day. A pharmaceutical with a long half life such as cabergoline or pergolide may be administered once per 1 to 10 days, for example. Alternatively, it may be administered 1 to 3 times per week.

In the case of using cabergoline with the longest half life, administration of a very small amount of 0.1 to 3 mg/week, or 0.1 to 0.5 mg/week shows an excellent effect of an agent for ameliorating a skin symptom such as senile plaque, a hair growth agent, or a slimming agent, and thus is useful. Furthermore, as the administration amount of cabergoline is very small, it is considered that there would be almost no side effect.

### EXAMPLES

Hereinbelow, the present invention is specifically described in detail based on Examples. However, Examples are given to specifically describe suitable embodiments of the present invention and the present invention is not limited only to Examples.

### Example 1

To find out the pharmaceutical effect of orally administered cabergoline, a change in the symptom of 5 senile plaques occurring on the back of both hands of patient A was investigated for 10 weeks from Jun. 20, 2013 (Thursday) to Aug. 29, 2013 (Thursday).

Patient A was a 70-year-old male with prolactin-producing pituitary adenoma. Before the clinical test of the treatment, patient A had dark brown circle-like senile plaques, specifically, 3 plaques on the back of the left hand (long diameter 11 mm×short diameter 8 mm, long diameter 8 mm×short diameter 6 mm, and long diameter 7 mm×short diameter 5 mm) and 2 plaques on the back of the right hand (long diameter 10 mm×short diameter 8 mm and long diameter 7 mm×short diameter 6 mm).

According to the doctor's instruction, oral administration was performed twice/week, on Monday and Thursday, for the first 4 weeks (from June 20 to July 15), and the dose was 2 mg/4 weeks. During this administration period, the state of the 5 senile plaques was directly observed, but no change was recognized.

Patient A was examined by the doctor on July 17 (Wednesday), the measured prolactin level in blood was 101.8 ng/mL, indicating a dramatic decrease from the level of 20888 ng/mL on April 18.

In view of the dramatic improvement of prolactin level, the doctor ordered that the cabergoline administration for the next 1 month be changed to one tablet/week, and oral administration be carried out before sleeping on Thursday. Furthermore, according to the examination on August 14, the prolactin level was decreased from 101.8 ng/mL to 47.8 ng/mL. As such, the cabergoline administration was maintained at one tablet/week, and the administration period was extended up to 2 months.

A total of 1 mg of cabergoline were orally administered by this 4-week administration (administration dates are as follows: July 18, July 25, August 1, and August 8).

In August and 6 weeks after starting cabergoline administration, it was observed that the color of the senile plaques started to become lighter.

Patient A was examined by the doctor on August 14 (Wednesday), and the measured prolactin level in blood was decreased from 101.8 mg/mL to 47.8 ng/mL. Thus, the treatment regimen using cabergoline from August 15 to October 3 was also determined to be oral administration of one tablet (0.25 mg) on Thursday.

The cabergoline administration on week 9 and week 10 was performed on August 15 (Thursday) and August 22 (Thursday).

From mid-August, senile plaques started to show a tendency to shrink in size. At the end of August, they clearly disappeared. From that time point, the back of the both hands, which had been flecked with pigmented macules and dry, gradually started to become white and have lighter skin texture, resulting in that the senile plaques on the back of the both hands were improved.

### Example 2

10 moles were present within an area having a width of 10 cm and a length of 8 cm extending from the outer side to the front side of the femoral region of the right leg. After 7 months, the state of those 10 moles was observed and the effect of oral administration of cabergoline was investigated. The results are shown in Table 1.

For the first 4 weeks from June 19 to July 17, one tablet (0.25 mg) of carbegoline was administered on Monday and Thursday. The total dose over the 4 weeks was 2 mg. For the following 26 weeks, one tablet of cabergoline was administered on every Thursday. The total dose of cabergoline over the 7-month treatment period was calculated to be 8.5 mg.

TABLE 1
Pharmaceutical effect of oral administration of novel skin treatment agent on 10 moles present in region
of width 7 cm × length 8 cm extending from upper right side to front side of femoral region of right leg

| No. Site | Size on Day Jun. 20, 2013 (mm) | Size on Day Jan. 20, 2014 (mm) |
|---|---|---|
| 1. Upper part of side area | 2.8 × 5.0 Black rectangle | 1.5 × 2.8 Dark brown rectangle |
| 2. Lower part of side area | 2.7 × 4.5 Black rectangle | 1.4 × 2.9 Faint, light brown rectangle |
| 3. Upper part of middle area | 2.8 × 2.8 Dark brown circle | 1.2 × 1.3 Faint, light brown circle |
| 4. Central left part of middle area | 2.1 × 1.6 Brown rectangle | 0.8 × 0.6 Light red circle-like |
| 5. Central right part of middle area | 2.3 × 2.1 Dark brown oval | 1.1 × 0.9 Red circle-like |
| 6. Lower part of middle area | 2.6 × 2.5 Dark brown circle | 1.3 × 1.3 Dark brown circle |
| 7. Upper part of front area | 3.3 × 3.1 Brown circle-like | Turned into weak pigmented macules having |
| | | flesh color with almost no change in size |
| 8. Right part of front area | 4.6 × 4.4 Light brown circle | Same as above |
| 9. Left part of front area | 1.2 × 1.1 Light brown circle | Same as above |
| 10. Lower part of front area | 1.5 × 1.4 Dark brown circle | Same as above |

Mole size: width mm × length mm

The state of the moles after 7 months, on Jan. 20, 2014, is shown in Table 1.

Among the two moles on the side area, the upper mole shrunk to a dark brown rectangular mole (2.8 mm×1.5 mm), and the lower mole shrunk to a faded light brown oval mole with an unclear border (2.9 mm×1.4 mm).

Meanwhile, the moles on the middle area were changed to red circle-like moles of 1.4 mm×0.7 mm and 1.1 mm×0.7 mm. At positions sandwiching the moles, 2 light brown circular moles with a diameter of 1 mm were recognized.

All of the 4 moles on the front area turned into faint flesh colored, pigmented macules, and they were no longer evident. The moles of 4.5 mm and 3 mm hardly shrunk and maintained almost the original shape. However, the remaining 2 small moles shrunk to circle moles with a diameter of 1 mm.

### Example 3

On the part slightly above the left ankle, 4 black moles were observed. From a direction of the little toe of the left foot, there were 3 black circular moles having a similar diameter ranging from about 3.7 mm to about 4 mm, present almost in one line with an interval of 2.4 cm and 2.6 cm. At a position 1.5 cm away from the third mole, there was a black oval mole of 3 ram×1.8 mm.

At a position 6 mm above the second mole, there was a red oval mole of 1 mm×1.7 mm. Meanwhile, at a position 17 mm above the third mole, 3 circular moles with a diameter of 1.5 mm, having black color, dark brown color, and red color, respectively, were present with an interval of 1 mm.

TABLE 2
Pharmaceutical effect of oral administration of novel skin treatment agent for 4 moles on left ankle that are present along
the line with length of 6.2 cm and 4 more small moles around them.

| Measurement | | | | |
|---|---|---|---|---|
| date | No. 1 | No. 2 | No. 3 | No. 4 |
| Jun. 20, 2013 | Black circular mole | Black circular mole with a | Black circular mole with a diameter | Brown circular mole of |
| | with a diameter of | diameter of 3.9 mm. | of 4.0 mm. | 2.0 × 1.8 mm. |
| | 3.7 mm. | | At 17 mm above, circular moles | |
| | | | with a diameter of 1.5 mm and | |
| | | | having black color, dark brown | |
| | | | color, and red color were present | |
| | | | in one line at an interval of 1 mm. | |
| Dec. 4, 2013 | The mole shrunk to | The mole shrunk to a light brown | The mole shrunk to a blackish | The mole shrunk to a |
| (after 166 | a red circular mole | oval mole of 2.0 × 2.4 mm with a | brown rectangular mole of 2.5 × 3.0 | yellow-brown circular mole |
| days) | with a diameter of | vague border. The black color | mm with a vague border. | with a diameter of 1.0 mm, |
| | 1.2 mm. The color | faded to light brown. | Moles at an upper part turned into a | and the brown color faded |
| | was changed from | Above the mole, a red oval mole | light brown oval mole of 1.0 × 1.5 | to yellow-brown. |
| | black to red. | of 0.6 × 1.2 mm was present. | mm and 2 red moles with a | |
| | | | diameter of 0.5 mm. | |
| Jan. 17, 2014 | Red circular mole | Light brown oval mole of 1.8 × | Blackish brown rectangular mole of | Light brown line-shaped |
| (after 210 | with a diameter of | 2.2 mm with a vague border. | 2.3 × 2.7 mm with a vague border. | mole of 0.1 × 0.3 mm. |
| days) | 1.0 mm. | The upper mole disappeared. | The upper 3 moles disappeared. | |
| Mar. 20, 2014 | Light red circular | Brown circular mole with a | Dark brown rectangular mole of | Very light brown line- |
| (after 272 | mole with a | diameter of 2.0 mm with a vague | 2.1 × 2.5 mm with a vague border. | shaped mole of 0.1 × 0.2 |
| days) | diameter of 1.0 mm. | border. | | mm. |

Mole size: width mm × length mm

On Mar. 20, 2014, 27 days after, the state of the above mentioned moles was investigated. The first mole on the left toe side had a red circular shape with a diameter of 1 mm and appeared to disappear. The second mole turned into a pale light brown circular mole with a diameter of 2 mm with a vague border.

The third mole had a blackish brown color and an almost rectangular shape of 2.1 mm×2.5 mm. Initially, those 3 moles were black circular moles of a similar size, but they followed a completely different path after the treatment.

Regarding the remaining forth mole, only a trace of a faint line shape was observed. It was recognized that the mole present above the second mole and the 3 moles present above the third mole completely disappeared after 210 days, on Jan. 17, 2014.

### Example 4

Investigation was made for the treatment of seborrheic keratosis generated from senile plaque. On the back side of the wrist of the right hand of patient A, there was seborrheic keratosis with width of 15 mm×length of 10 mm, which had remained as a problem for 13 years. In summer, elevation of the skin of about 4 mm with blackish brown color occurred. By the oral administration of cabergoline over the last year, the elevation of the skin became flattened and the color faded from blackish brown to brown.

Furthermore, 2 years ago, seborrheic keratosis with width of 13 mm×length of 9 mm was caused from senile plaque on the back of the right hand, and elevation of the skin of about 2 mm with dark brown color was recognized. However, by the pharmaceutical effect of cabergoline, it shrunk to a size of width 9 mm×length of 5 mm as of March 22, and the color faded to pale yellow brown, like flesh color. Thus, it is recognized that the seborrheic keratosis with a short clinical history is more responsive to the treatment.

### Example 5

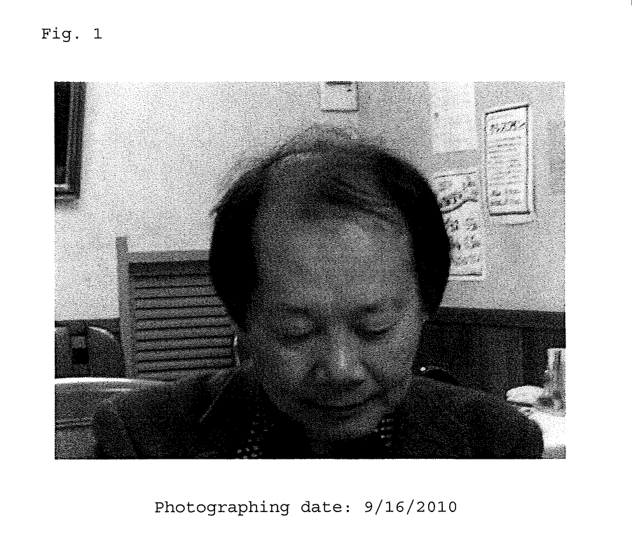

According to the photograph (1) of patient A (FIG. 1) taken at a restaurant during travel in Shonai region of Yamagata Prefecture on Sep. 16, 2010, the head of patient A from the upper forehead to the crown was quite bald. Only a small amount of hair was left right above the forehead. Patient A was 67 years and 2 months old at the time of taking the photo. Since the cabergoline administration was started on Jun. 20, 2013, patient A was 69 years and 11 months old at that time, which was 2 years and 9 months after taking the photograph (1), and thus, aging further progressed so that the hair became thinner and the hair loss progressed.

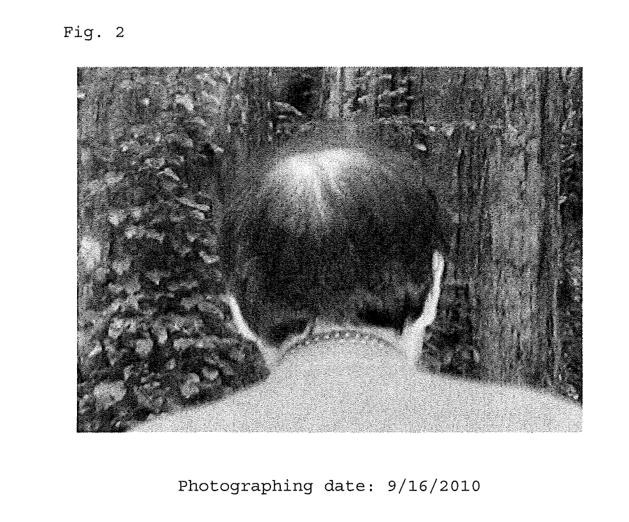

The image of the occipital region of patient A taken from the back side on Sep. 16, 2010, is shown in the photograph (2) (FIG. 2).

In the period from Jun. 20, 2013 (Thursday) to Jul. 15, 2013 (Monday), one tablet of 0.25 mg cabergoline was orally administered twice per week, on Monday and Thursday, and thus a total of 2 mg of cabergoline was administered over one month. From July 18, the administration of cabergoline was changed from two tablets per week to one tablet per week. The total dose of cabergoline administered from Jul. 18, 2013 to Jan. 20, 2014 was about 6 mg, and thus, the total amount of cabergoline administered after the start of administration was calculated to be 8 mg.

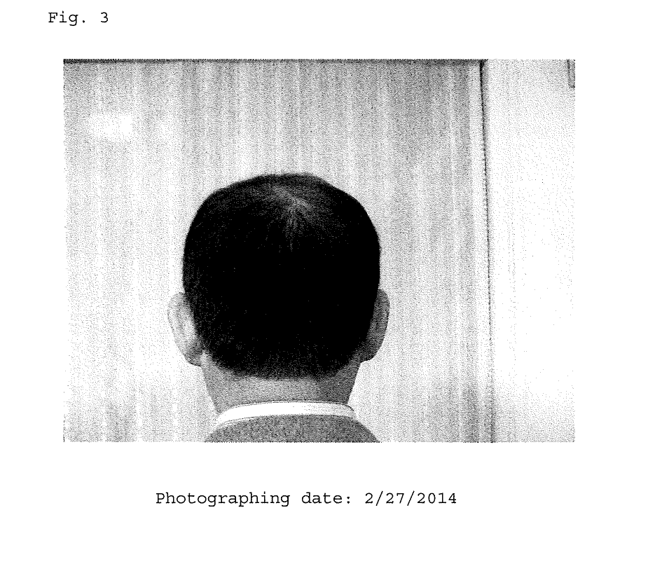

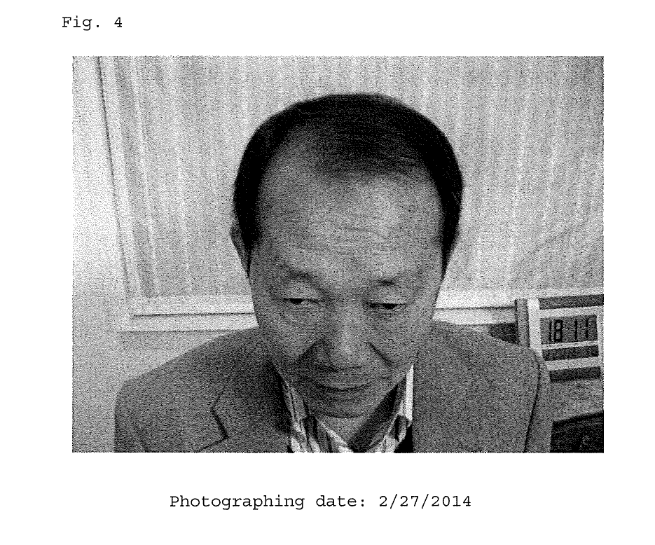

Images of the frontal region and the occipital region of patient A taken on Feb. 27, 2014 are shown as photograph (3) (FIG. 3) and photograph (4) (FIG. 4), respectively.

As shown in the photograph (3), short black hairs were found in the previously bald region, and the bald area was clearly reduced.

When the photograph (2) of the occipital region of patient A taken on Sep. 16, 2010 was compared to the photograph (4) of the occipital region of patient A taken on Feb. 27, 2014, it was found that the bald area was reduced in the region from the crown to the occipital region of patient A after the oral administration of cabergoline. Thus, it is evident that, like hairs on the frontal side of the head, hairs were increased in the region from the crown to the occipital region.

### Example 6

Patient A had attempted to lose weight by the combination of restricted diet and exercises including walking, walking up and down stairs, and abdominal muscle exercise. The body weight had been reduced to 56.5 kg within about 2 months twice. However, in those cases, once the body weight was reduced to about 57 kg, hungry feeling was felt so powerfully and then the body weight was gained back to the initial one after it reached about 56.5 kg.

Due to an illness, patient A had to undergo the cabergoline treatment from the end of June of year 2013, and for this reason, attempted to lose weight again. Since it was expected that the body weight would be gained back, the target value was set at 56.5 kg, and a long-term plan without any exercise or restricted diet was adopted. However, regular meals were taken and only the overeating was strictly avoided.

Contrary to expectation, the body weight was steadily reduced to the target value. There was a concern about gaining back the weight, but the concern did not come true.

There was no hungry feeling this time. In this regard, it is considered that the hungry feeling is not caused at all as cabergoline not only contributes to the lipolysis in the abdomen area but also probably works on the brain center. The waist size was reduced from 91 cm at a maximum to 83 cm. It is considered that the mechanism for the body weight loss is based on the degradation of lipids in adipocytes which is caused by binding of cabergoline to D₁ and D₅ dopamine receptors to increase cAMP and Ca²⁺ in the adipocytes. It should be noted that the normal BMI for the Japanese aged 70 and above is 21.5 to 24.9 (in year 2015).

It was confirmed that, in the case of the oral administration to patient A, BMI was decreased to a normal level after 8 weeks.

TABLE 3
Carbergoline: example showing slimming effect by administration of 0.25
mg/week Administration period: Sep. 5, 2013-Dec. 12, 2013 (15 weeks)

| Date of | Body weight | Weight loss | Total weight loss | Obesity |
|---|---|---|---|---|
| measurement | (kg) | (kg) | (%) | BMI |
| Sep. 5, 2013 | 62.3 | | | 26.6 |
| September 19 | 61.3 | 1.0 | −1.6 | 26.2 |
| October 3 | 60.1 | 1.2 | −3.5 | 25.7 |
| October 17 | 59.1 | 1.0 | −5.1 | 25.2 |
| October 31 | 58.3 | 0.8 | −6.4 | 24.9 |
| November 14 | 57.5 | 0.8 | −7.7 | 24.6 |
| November 28 | 56.8 | 0.7 | −8.9 | 24.3 |
| December 12 | 56.1 | 0.7 | −10.0 | 24.0 |
| Mar. 14, 2015 | 54.2 | 1.9 | −13.0 | 23.2 |

Meanwhile, since cabergoline was administered at 0.25 mg/week for 3 months of the measurement period, the total dose of cabergoline was calculated to be 3 mg. Since the maximum daily dose of cabergoline allowed for a patient with Parkinson's disease is 3 mg, it is surprising that a desired body weight loss was achieved with a total amount of 3 mg of cabergoline. In this regard, it is considered that no side effect would appear with administration of such a small amount.

1-15. (canceled)

16: A method for ameliorating a skin symptom, comprising administering an effective amount of a dopamine agonist, wherein the skin symptom is selected from the group consisting of a pigmented macule, a periorbital dark circle, darkish skin, black darkish skin, a mole, an ephelis, a birth mark, and seborrheic keratosis.

17: The method according to claim 16, wherein the dopamine agonist is at least one kind selected from the group consisting of cabergoline, pergolide, bromocriptine, talipexole, pramipexole, ropinirole, rotigotine, apomorphine, terguride, aripiprazole, and a salt thereof.

18: The method according to claim 16, wherein the dopamine agonist is orally administered.

19: The method according to claim 16, wherein the dopamine agonist is cabergoline and an effective amount thereof is 0.1 to 3 mg/week.

20: The method according to claim 16, wherein the dopamine agonist is cabergoline and an effective amount thereof is 0.1 to 0.5 mg/week.

21: The method according to claim 16, wherein the dopamine agonist is at least one kind selected from the group consisting of cabergoline, pergolide, bromocriptine, terguride, and a salt thereof.

22: The method according to claim 16, wherein the dopamine agonist is at least one kind selected from the group consisting of cabergoline and a salt thereof.

23: The method according to claim 17, wherein the dopamine agonist is orally administered.

24: The method according to claim 19, wherein the dopamine agonist is orally administered.

25: The method according to claim 20, wherein the dopamine agonist is orally administered.

26: The method according to claim 21, wherein the dopamine agonist is orally administered.

27: The method according to claim 22, wherein the dopamine agonist is orally administered.
